(19) United States
(12) Patent Application Publication
(10) Pub. No.: US 2026/0031226 A1
(43) Pub. Date: Jan. 29, 2026

(54) SYSTEMS AND METHODS FOR ALARM RESPONSE

(71) Applicant: Welch Allyn, Inc., Skaneateles Falls, NY (US)

(72) Inventors: John A. Lane, Venice, FL (US); David E. Quinn, Auburn, NY (US); Tyson B. Whitaker, Arden, NC (US); Gene Wolfe, Pittsford, NY (US); WonKyung McSweeney, Manlius, NY (US); Danielle Endres, Syracuse, NY (US)

(73) Assignee: Welch Allyn, Inc., Skaneateles Falls, NY (US)

(21) Appl. No.: 19/280,918

(22) Filed: Jul. 25, 2025

Related U.S. Application Data
(60) Provisional application No. 63/676,274, filed on Jul. 26, 2024.

(51) Int. Cl. G16H 40/63; A61B 5/00; G06T 7/70

(52) U.S. Cl. CPC G16H 40/63; A61B 5/742; A61B 5/746; A61B 5/748; G06T 7/70; G06T 2207/30004

(57) ABSTRACT

Methods and systems for providing context for alarms and resolving alarms using a virtual agent are described. Inputs including imaging, patient information, and aural, visual, and mechanical patient inputs are analyzed to provide context for an alarm. Based on the context, the methods and systems may identify a patient action that may resolve the alarm. A virtual agent may interact with the patient to identify and resolve the alarm without additional caregiver input.

### CROSS-REFERENCE TO RELATED APPLICATION

This application claims priority to and benefit of U.S. Provisional Patent Application No. 63/676,274 entitled SYSTEMS AND METHODS FOR ALARM RESPONSE and filed Jul. 26, 2024, the contents of which are incorporated herein by reference in their entirety.

### TECHNICAL FIELD

This application relates generally to responding to and resolving medical device alarms. Specifically, this application relates to the use of an interactive virtual agent to assist in responding to alarms.

### BACKGROUND

In the United States, there are an average of 15-20 medical devices per hospital room emitting 350 alerts per bed per day (Jones K. Alarm fatigue a top patient safety hazard. CMAJ. 2014 Feb. 18; 186(3):178). Given the arrangement of rooms, the number of patients being tracked, and the volume of the alarms, it is challenging for healthcare providers to respond to every alarm in a timely manner. Further, 80%-99% of alarms in hospital units are false or clinically insignificant, leading to alarm fatigue and desensitization (Fernandes C, Miles S, Lucena CJP. Detecting False Alarms by Analyzing Alarm-Context Information: Algorithm Development and Validation. JMIR Med Inform. 2020 May 20; 8(5):e15407). Alarms may be difficult to distinguish from each other and provide little context to a provider, leading to desensitization, increased response times, and worsening patient outcomes.

### SUMMARY

Provided are methods and systems for addressing alarm conditions with minimal or no caregiver input, and methods and systems for prioritizing alarms and efficiently conveying appropriate information to a third party if needed. Using an interactive system and analysis of image data of a patient area containing one or more medical devices at least partly disposed within a patient area, the cause of an alarm and an action to resolve the alarm may be identified. The system may activate a virtual assistant to interact with a patient to obtain additional information or request a patient action to resolve the alarm. In the event a patient is unable to resolve an alarm, a second virtual assistant may be activated to interact with an in person or remote caregiver. The use of the second virtual assistant may allow for streamlining of the patient load, responsiveness, and improved care.

In some aspects, a computing device may receive an indication of an alarm from a device associated with a patient. Image data, including images of the device and patient, acquired continuously, periodically, or episodically from one or more imaging device(s) may be analyzed to determine the cause of the alarm. Imaging devices may include, for example, an RGB sensor, a digital camera, an infrared camera, a thermal camera, a depth imaging device, a 3D time of flight camera, a LIDAR camera, or other camera types.

Image data may be captured at single or multiple points in time. In some aspects, image data captured at multiple different points of time may be compared to determine the cause of the alarm. In other aspects, a single image is sufficient to determine the cause of the alarm. Based on the cause of the alarm, the system and methods may be used to determine a next step that may resolve the alarm. Such a next step may include obtaining information from the patient, and/or requesting that the patient perform a specific action.

In some aspects, the specific action may be determined using, for example, a rule database, machine learning, a large language module, generative artificial intelligence, and the like. In some aspects, data included in the determination may be obtained by analyzing image data and information in one or more databases including alarm protocols, device instructions, device operation manuals, hospital protocols, electronic medical records, sensor data, medical device data, and the like. The system may generate an interactive virtual agent configured to visually or audibly present to the patient a request for additional information and/or the performance of a specific action to resolve the alarm. A patient may respond to the virtual agent using, for example, a physical or audible response in an effort to address the cause of the alarm. In the event that a patient is unable to resolve an alarm, a second virtual assistant may be generated and interact with the caregiver. The second virtual assistant may contact the caregiver, present the relevant patient information to the caregiver, and, based on the instructions received from the caregiver, resolve the alarm. In some aspects, the instructions from the caregiver may be that the caregiver is going to go to the patient's room. In other aspects, the instructions may provide for remote actions that can be taken by the system. In some aspects, the second virtual agent can prioritize any one alarm in a series of alarms either independently or via instructions from the caregiver.

To the accomplishment of the foregoing and related ends, certain illustrative aspects of the system are described herein in connection with the following description and the attached drawings. The summary is provided to introduce a selection of concepts in a simplified form that are further described below in the Detailed Description. This summary is not intended to identify key features or essential features of any subject matter described herein.

### DESCRIPTION OF THE FIGURES

The following figures, which form a part of this disclosure, are illustrative of the described technology and are not meant to limit the scope of the claims in any manner.

FIG. 1 illustrates a schematic block diagram of an example alarm management system according to an embodiment.

FIG. 2 illustrates an example system architecture for an alarm management system according to an embodiment.

FIG. 3 illustrates a top down view of an example alarm management system environment according to an embodiment.

FIG. 4 illustrates a top down view of an example alarm management system environment according to an embodiment.

FIG. 5 illustrates exemplary alarm context data according to an embodiment.

FIG. 6 illustrates a method for using a virtual agent to resolve an alarm according to an embodiment.

FIG. 7 illustrates a method for evaluating an alarm according to an embodiment.

FIG. 8 illustrates a method for using a plurality of virtual agents to resolve an alarm according to an embodiment.

FIG. 9 illustrates a machine learning model according to an embodiment.

FIG. 10 illustrates a block diagram of a computing system, according to at least one example.

### DETAILED DESCRIPTION

Various implementations of the present disclosure will be described in detail with reference to the drawings, wherein like reference numerals present like parts and assemblies throughout the several views. Additionally, any examples set forth in this specification are not intended to be limiting and merely set forth some of the many possible implementations.

Provided is a system and method for remotely obtaining additional information about an alarm and using one or more virtual agent(s) to communicate with a patient and/or caregiver and address an alarm condition without always requiring caregiver input or presence. A virtual agent may communicate using sound, images, or a combination thereof. In some examples, the systems and methods described herein acquire information from a patient or about objects within a patient's room and, utilizing a decision module, determine an action to be taken. A decision module may use any of a variety of technologies with one or more information sources. For example, the decision module may include a rule database, machine learning, a large language module, generative artificial intelligence and the like. In some aspects, the data in the decision module may include information in one or more databases including alarm protocols, device instructions, device operation manuals, hospital protocols, patient information, electronic medical records, sensor data, and the like.

Information regarding a patient and a patient's room may be collected from audial, visual, or mechanical inputs. The various inputs may be analyzed, for example, using machine learning, and the appropriate response may determined by inputting extracted context into a decision module. The decision module may output an action such as alerting a clinician, providing an answer to a patient, asking a patient for additional information, providing instructions to a patient, setting or re-setting alarms, making notes in an electronic medical record, and the like.

In some aspects, when an alarm sounds or a patient initiates contact, the system may attempt to acquire additional information. Additional information may come from image data, networked devices, medical records, spoken words, mechanical inputs, sensors, and images of the patient, medical devices, and patient room.

For example, an imaging device may acquire an image of the patient or objects within the patient's room. Exemplary imaging devices for collecting patient input may include an RGB sensor, a digital camera, an infrared camera, a thermal camera, a depth imaging device, a 3D time of flight camera, a LiDAR camera or other camera types that may be used to capture image data in one or more wavelength ranges. In some aspects, image data may be acquired using a depth imaging device that can generate distance from the imaging device to objects within its field of view in which the intensity of the pixel corresponds to the distance from the camera. In some aspects, the imaging data may be used to generate point clouds where each pixel in the depth image corresponds to a point in 3D space, defined by its x, y, and z coordinates. This allows for the reconstruction of the 3D geometry of the scene, enabling applications such as 3D modeling, object detection, and augmented reality.

In some aspects, imaging devices may include a digital camera configured to capture video and/or still images (e.g., digital photographs) of/depicting the patient, medical devices, and/or other items and people within a field of view of the camera. In some examples, the imaging device may include image-altering features such as pan, tilt, and zoom.

Images may be analyzed, for example, using machine learning to identify the objects, including patients, caregivers, and clinicians within the room. Identification of objects including patients and other individuals within the patient room of the care facility enables the system to determine changes to the objects within images alone or in conjunction with other inputs such as sound, machine data, patient input, medical records, hospital databases, and sensor data, and, using machine learning models, provide context for a detected alarm that can be used to resolve the alarm.

Using the context of the detected alarm, the system may determine an appropriate action. The appropriate action may be determined by a variety of means. In some aspects, the system may access a rule database and identify a specific rule that applies to a situation. In other aspects, the system may use generative AI to provide an answer. In other aspects, machine learning may be used to identify the appropriate action. In some aspects, a combination of rules, generative artificial intelligence (AI), and machine learning may be used to determine the appropriate action. The identified action may be sent to any combination of devices, virtual agents, or caregivers. In some aspects, the virtual agent may interact with a patient to obtain more information or to instruct the patient to perform specific actions to address the alarm. In other aspects, if a patient is unable to resolve an alarm, a second virtual agent may be initiated to interact with a caregiver. The second virtual agent may present the caregiver with information about the patient, status of the patient, cause of the alarm, and the like, allowing the caregiver to provide instructions to the second virtual agent regarding how to resolve the alarm. Such instructions may include actions that can be taken by the system, additional personnel to notify, or notification that the caregiver will be entering the room. In some aspects, the virtual agent may prioritize the alarm based on the context and/or instructions received by the caregiver.

In some aspects, the extracted data may identify individuals and objects within a patient area, as well as the condition, placement and relative positioning of such individuals and objects. For example, if an alarm sounds and the system determines that a caregiver is in the room and facing the device sounding the alarm, the system may determine that further intervention by the alarm management system is not necessary as the caregiver is addressing the issue. In other aspects, if the patient is alone, or the caregiver is occupied in another area of the room, the system may determine that additional steps should be taken to address the alarm including activating a virtual agent to interact with the caregiver and/or the patient. Through the use of a decision module which may include an alarm rule base and/or generative AI, the system may triage alarms, identify critical and non-critical alarms, and use a virtual agent to interact with a patient to resolve alarms that do not require caregiver intervention.

In some aspects, the virtual agent may interact with a patient at episodic or periodic moments of time that are not initiated by any alarm or event. For example, the virtual agent may ask the patient for information about their condition, pain levels, and the like. The information may be entered into the patient record or forwarded to a caregiver for further action.

In some aspects, the system may instruct the virtual agent to interact with a patient in anticipation of an event. For example, the system may continuously monitor a patient, gathering information as to patient condition, patient position, pain levels, or other relevant aspects of patient care. In the event that the system determines there is a patient condition that could trigger an alarm, the system may initiate the virtual agent to instruct the patient to take action. For example, if the patient has been instructed to lay on their side or keep their head elevated and the patient is no longer doing so, the virtual agent may instruct the patient to change position. If the patient is positioned such that a reading cannot be taken, the virtual agent may instruct the patient to change position so that the reading may be acquired. These instructions and interactions may take place even though an alarm has not sounded. In some aspects, the system may determine that action needs to be taken even if it is not currently predicting an alarm. For example, if an IV line is positioned such that it is not flowing correctly, but is flowing above a threshold amount that would trigger an alarm, the virtual agent may instruct the patient to reposition their body to restore normal flow.

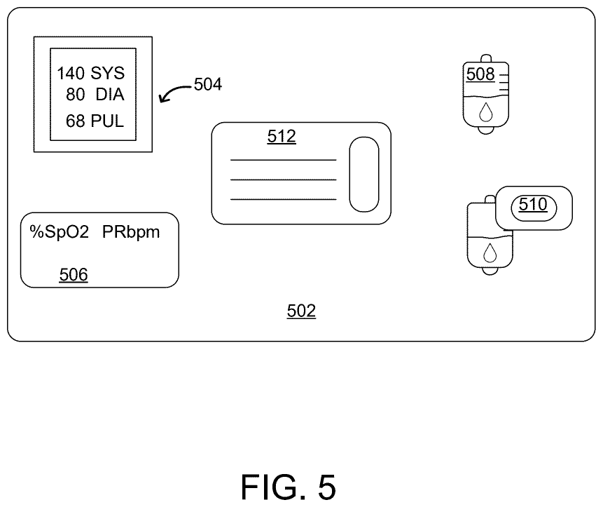

In some examples, the system may automatically identify individuals in a room based on visual characteristics, such as using facial recognition and/or identification of a visual or electronic marker (such as an ID badge or patient bracelet) of the individual. Additionally, the systems may be used for identifying objects, such as medical devices, treatment devices, medications, sensors, and other objects as well as the state or settings of those objects within the room of the patient and any interactions between the objects and the patient. The system may record data based on the identification indicating, for example, when treatments are administered, settings for treatments, when treatments finish, and other contexts for the alarm. In some aspects, images acquired from a patient room may be converted to cartoons or pictographs as shown, for example, in FIG. 5 in which information obtained from one or more devices or images within the room is presented on a display.

The computing device may determine, using one or more inputs and one or more machine learning models, an event occurring within the room. The machine learning model may be trained using training data including annotated images of patient care with annotations of event data as well as annotated sensor data. In some examples, a first machine learning model may be used to identify events within the room, a second machine learning model may be used for object detection of relevant objects associated with the events (e.g., equipment used or interacted with), a third machine learning model may identify individuals based on enrollment data, and a fourth machine learning model may provide context for an alarm based on the identified event, object, and people in the room.

In some aspects, vocal input from the patient may be used alone or in conjunction with the image data and/or sensors to provide context for the alarm. A fifth machine learning model trained on diverse speech data may use natural language understanding, hidden Markov models, gaussian mixture models, deep neural networks, recurrent neural networks, connectionist temporal classification, convolutional neural networks, transformer-based models, transfer learning, beam search decoding, speech synthesis, and/or text to speech to extract meaning from vocal input. Based on the context, the system may analyze the resulting context using a decision module including a rule database and/or generative AI to determine an action to resolve the alarm.

The devices within a patient's room may, in some examples, be equipped with communication modules capable of transmitting data to the computing system. In this manner, medical devices may provide data, including for example pulse, blood oxygenation, therapeutic drug delivery rate and amount, blood pressure, or any other suitable information. Along with transmitted data, information from devices may additionally or independently be collected using the microphone, sensors, mechanical input, and one or more imaging devices present in the patient's room. For example, a medical device having a display may be visible to the camera and a computing device may be configured to extract data from the medical device based on the information displayed on the display of the device. Various devices may also have lights or other notifications that indicate a condition of the device. For example, a light may indicate if the machine is on or off, or if it is functioning or not functioning as expected.

Images may be parsed by the system to identify the current state of the device. For example, images may be parsed to identify each specific object and the relation of each object to other objects in the room. For example, if an alarm sounds on one device, one or more of the other devices in the room may be evaluated and context may be provided in terms of the collection of devices or other objects in the room. For example, if the SPO₂ decreases, the images may be analzyed to identify the presence or absence of an oxygen mask and if the mask has been disloged, the system may instruct the patient to replace the oxygen mask. In other aspects, the system may identify an alarm as an error or false alarm and may therefore wait to notify a caregiver until a later time as the system has determined that it is a low priority alarm. In some aspects, the system may help identify the acuity of a patient's condition based on the number and type of alarms that are sounded for a particular patient, allowing for better allocation of resources.

In some aspects, the system may take into consideration the history of the device. For example, when the device was started or connected to the patient, if the device was turned on, and/or when it was last manipulated by a care provider. Using the history of the object, the system may identify the current state of the device, apply a model of what the device does, and identify a likely error that triggered the alarm from a set of possible errors. For example, the model may include what “on” looks like, what “off” looks like, what “done” looks like, normal activity, types of errors, sources of errors, and the like.

After an alarm is identified by the system, regardless of whether the device sounding the alarm is networked, the system may use collective data from the device sounding the alarm and other devices or objects including people in the room as well as other information sources such as databases to provide a context for the alarm. In some aspects, the system and methods may determine that patient action will resolve the alarm. For example, if the patient is positioned such that a infusion line has become kinked, or the patient is in a position that cannot be monitored, a virtual agent may provide instructions to the patient to resolve the issue.

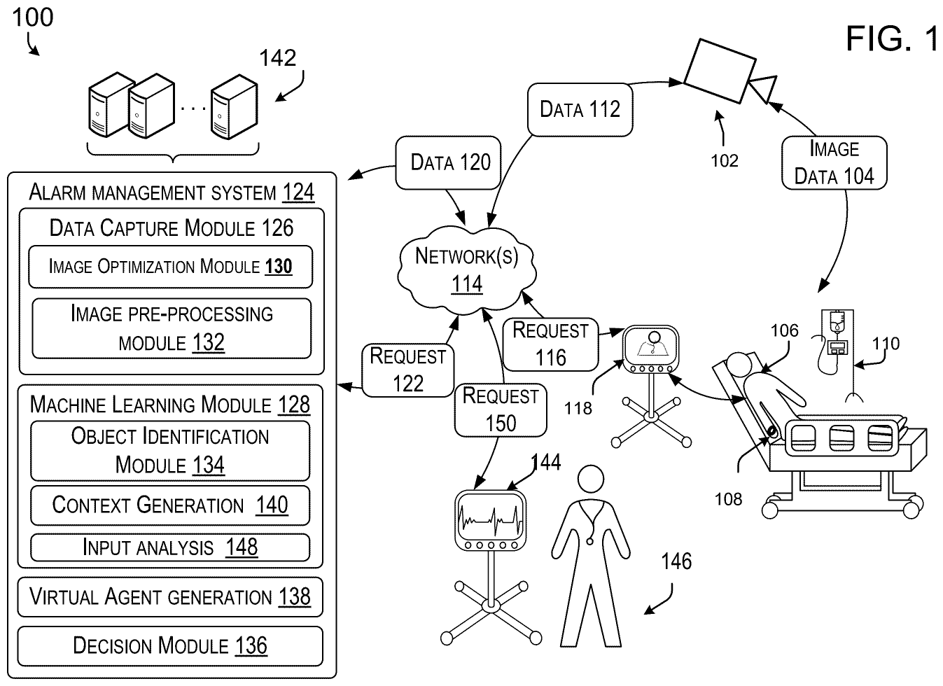

For example, the system may record when a bag was attached to an intravenous (IV) line of an infusion pump as well as the level of the contents of the IV bag. The system may analyze an image at a later point in time to identify the infusion pump, for example, infusion pump 110 of FIG. 1, and, using machine learning, determine a state of the infusion pump such as whether the IV bag is full, empty, or the rate of infusion. In some aspects, the state is determined by comparing an image to a prior image of the same IV bag or by comparing an image to a model of different IV states. For example, in a first image the bag attached to the IV line may be full. An alarm may sound and a second image may be captured. The second image may be compared to the first image using machine learning to identify any change in state with the IV bag. For example, the system may identify that the IV bag is now empty, that it is not emptying at the expected rate, or that the line has pulled out, or something else is different about the IV bag in the second image in comparison to the first image. Thus, when an alarm from an infusion pump sounds, the system may determine the state of the IV and the IV bag and access a decision module. For example, if a rule database is used, the system may identify the relevant rule for the alarm. In the event there is no rule, the system may notify a caregiver of the alarm. In some aspects, the system will provide context to the caregiver as to the cause of the alarm. If there is a rule, the system may execute the rule, for example by initiating a virtual agent to communicate with a patient. In some aspects, the alarm may be resolved by patient action. For example, if a line from the IV is identified as compressed, the patient may be instructed to change position in order to address the issue. In other examples, generative AI may be used. For example, a neural network may be trained using supervised or unsupervised learning to identify causes and potential resolutions for alarms. The system may be prompted with the alarm, analyze the context of the alarm, and return information as to how to resolve the alarm.

In some examples, the determinations and computations described herein may be performed locally, at a computing device local to the patient room such as on the device itself, or in a separate computing device in the room, and conveyed to a care provider device or to a single computing system where all patient data is processed. In some examples, the patient data may be anonymized and/or encrypted when processed by the computing device, with a code, key, or tag, that may be used to decode the patient identifier information after processing. In this manner, the patient privacy and security of health data may be preserved.

FIG. 1 shows a schematic block diagram of an example alarm management system environment 100 used to monitor patients. The example alarm management system environment 100 includes at least one data collection device 102 such as an imaging device and an alarm management system 124 in communication via one or more networks 114. While data collection device 102 is depicted as an imaging device, the device may include or represent other or additional forms of information capture such as spoken or mechanical inputs. The alarm management system environment 100 may additionally include other objects such as one or more medical device(s) as represented by infusion pump 110 and clinician display device 144, and response devices such as virtual agent 118. While FIG. 1 displays a sensor 108 attached to the patient 106, in some aspects the sensor may be located at one or more other regions of the body. Sensors may provide additional and/or confirmatory information to the alarm context system, allowing for further differentiation or confirmation of the information extracted from image or other data.

The alarm management system 124 may be part of or use one or more server computing devices and servers 142, which may communicate with data collection device 102, display device 144, and virtual agent 118 to send and respond to queries, receive data, act on data, and so forth. The alarm management system 124 may include one or more database systems accessible by a server storing different types of information. For instance, a database can store correlations and algorithms used to manage the imaging data, signal data, and other patient data to be shared between the data collection device 102, the virtual agent, and/or the display device 144. A database can also include clinical data, patient records, device records and the like. A database may reside on a server of the alarm management system 124 or on separate, remote computing device(s) accessible by the alarm management system 124.

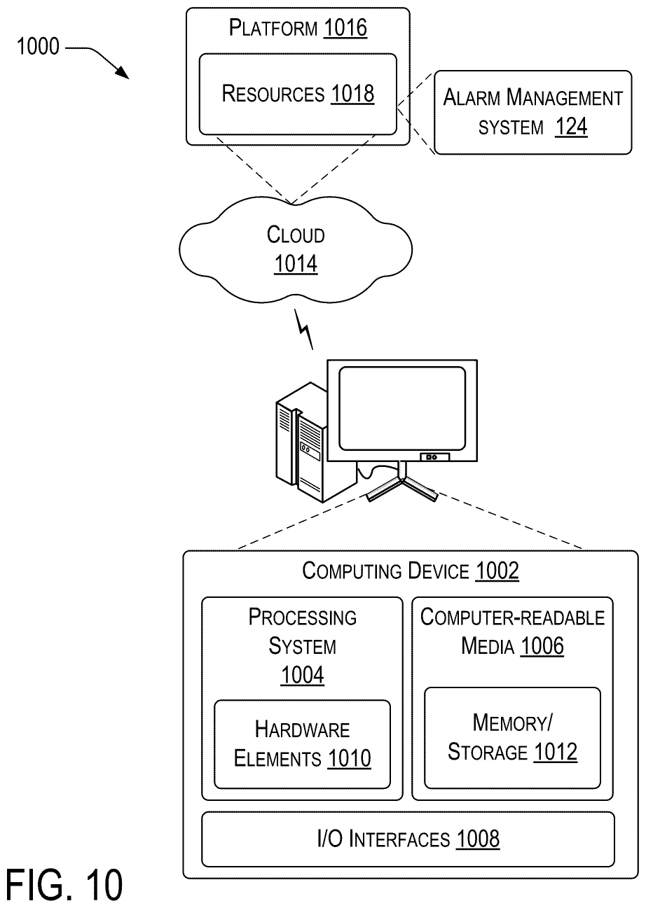

Communication between the computers and servers 142 of the alarm management system 124, the data collection device 102, the virtual agent 118, and/or the display device 144 can include imaging data, sensor data, and/or patient data related to the health of the patient such as EMR data. A server or other computing device of the alarm management system 124 may act on requests from the data collection device 102 received via data 104, and/or the display device 144 as received by request 150, determine one or more responses to these queries, and respond to the data collection device 102, and/or the clinician's display device 144 through the network 114. In some aspects, the system may send requests such as request 122 asking for information from the clinician device and/or one or more of the imaging device such as the data colletion device 102 and medical device such as infusion pump 110. For example, the system may request instructions from the clinician's display device 144, and therefore the clinician as to what images to capture or what information to obtain from the images. In other aspects, there may be pre-set or rule based determininations as to what type of information to extract from the images. A server of the alarm management system 124 may also include one or more processors, microprocessors, or other computing devices as discussed in more detail in relation to FIG. 10.

The network 114 is typically any type of wireless network or other communication network known in the art. Examples of network 114 include the Internet, an intranet, a wide area network (WAN), a local area network (LAN), a virtual private network (VPN), cellular network connections, and connections made using protocols such as 802.11a, b, g, n and/or ac. Alternatively or additionally, network 114 may include a nanoscale network, a near-field communication network, a body-area network (BAN), a personal-area network (PAN), a near-me area network (NAN), a campus-area network (CAN), and/or an inter-area network (IAN).

In some examples, the data collecting device 102 may include any device having imaging capabilities capable of capturing images of an object in the environment, such as a healthcare setting or a patient's room in a home environment. For example, the idata collecting device 102 may include a camera, such as an RGB sensor, a digital camera, an infrared camera, a thermal camera, a depth imaging device, a 3D time of flight camera, a LiDAR camera, or other such imaging device to name a few non-limiting examples. In some examples, the data collecting device 102 may include a device capable of capturing still images. Additionally or alternatively, the data collecting device 102 may include a video camera that may be capable of capturing a stream of imaging data. In some aspects, the imaging device may be connected to a microphone. In some aspects, the video system may transmit images to a clinician device or central station such as display device 144. Such devices may be one way or two way, transmitting an image of the room, or allowing a clinician to talk to or view a patient in the room.

A display device such as device 144 may provide visual and/or audio information. For example, the display device 144 may be a device with a display such as a computer or tablet, a wearable device such as a watch, phone, or other handheld device, or an audio only device such as an earpiece. In some aspects, the display device 144 may be secured such that only specific caregivers have access or a code or other security mechanism is in place to limit access to the device. In some aspects, the display device 144 may be a panel or screen in one or more rooms including a patient's room. In some aspects, the images may be converted to pictographs or other representative images, preserving a patient's privacy, but allowing relevant information to be conveyed as shown in FIG. 5.

The data capture module 126 may initiate image acquisition via a data collecting device 102 such as an imaging device. In some aspects, image acquisition may be initiated based on requests from clinicans or other individuals or objects associated with a patient. For example, a clinician may input a request into display device 144 and the request may be conveyed via request 150 to network 114 and then transmitted as request 122 to the alarm management system 124. In other examples, the protocol for the virtual agent 118 may initiate a request for additional information from the alarm management system 124 via request 116 to network 114 transmitted as request 122 to the alarm management system 124. Such requests may initiate one or more actions by the alarm management system 124 including image acquisition, processing, machine learning application execution such as machine learning from machine learning module 128, and decision resolution from decision module 136 that may access a rule database or generative AI. In some aspects, the data collecting device 102 or other sensor may detect the environment and use optimal enhancement to acquire the requested information. For example, the data collection device 102 such as an imaging device or other sensor may determine that the light in a room is dim. The lighting may then be adjusted to a state in which the desired information may be captured. In other aspects, the data collection device 102 duch as an imaging device may use different filters, lighting, or capture rates to acquire the desired image. In some aspects, the data collection device 102 such as an imaging device may use one or more types of imaging such as infrared imaging, thermal imaging, and the like. The type of image acquired may be based on one or more parameters of the room including the amount of light available, the time of day, the condition of the patient, the type of information being captured, and the like.

In some aspects, image acquisition may take place automatically in a continuous monitoring environment or may be stopped and/or started at a particular cadence or via a request, such as a request 150 from the clinician. In some aspects, it may start in response to an alarm. Captured image data 104 may be sent via network 114 to alarm management system 124 as shown via data 112 and data 120. Raw images or pre-processed images may be analyzed to detect the position of the patient as well as mechanical movement and light absorption or reflection from the patient. Pre-processing may be any form of image optimization or calibration performed using one or more devices in or connected to alarm management system environment 100. For example, the alarm management system 124 may input the image data into an image optimization module 130 which may alter the image data such that the image data is optimized to be at the highest quality. Such an optimization process may automatically assess the image data and adjust the image data to increase the resolution of the image data, re-format the image data into a correct format, re-size the image data to a correct dimension, or compress the image data, to name few non-limiting examples. In some aspects, the optimization system may crop the image to the area of interest such as a capture point. Thus, by optimizing the image data, the alarm management system 124 may obtain more accurate images, thereby more accurately identifying the object(s) in the image data. For example, based on capturing the image data, the imaging device may send the image data to imaging pre-processing module 132 of the alarm management system 124.

The images or sets of images are then analyzed via machine learning. In some examples, the machine learning module 128 may include a machine learning model trained to identify one or more objects in image data. In some aspects, the object identification module 134 may identify the patient, for example through facial recognition, scanning of an object such as a barcode on the wrist of a patient, or location beacons or other sensor systems associated with the object such as a radio frequency identification system (RFID). In some aspects, an object classification model may be used. In some aspects, the imaging device and/or alarm management system 124 may identify an object to be tracked throughout a series of images via object identification module 134. For example, a particular device or a person other than a patient such as a caregiver may be identified. In some aspects, the image may be pre-processed using a position estimation model to identify a patient orientation and position or the orientation or position of someone else in the room, for example a family member sitting in a chair or a caregiver interacting with objects in the room. In some aspects, range imaging and/or pressure mapping may be used to identify a patient orientation and position. Object identification may be combined with other inputs such as mechanical, sensor, or aural inputs. In some aspects, the other inputs are analyzed via input analysis 148 prior to being combined with object identification to generate context via context generation module 140.

The context generation module 140 may provide a context for the alarm. Each object in the image may be identified by the alarm management system 124. The alarm management system 124 may extract information regarding the object, the history of the object, and/or the state of the object transmitting the alarm. The information obtained about an object may be processed independently or in conjunction with an associated group of objects. For example, analysis of an area of an image including a vital signs monitor may provide information regarding sensor placement, patient activity/movement, and/or vital signs detected. Image analysis may be triggered by an alarm, or by a change in state of the room or of the object. For example, if a care provider enters or leaves the room, all objects in the room may be re-evaluated to determine if there has been a change of state, that is determinations as to whether a device has been turned on or off, if an object has been replaced or new objects added, and the like. If an alarm sounds, the image of the vital signs monitor alone or in combination with readings from and images of other sensors or devices may be analyzed to provide context for the alarm. Any type of image analysis may be used. Context may be detected using, for example, joint detection, object detection, facial landmark detection, and non-contact vital detection. For example, an alarm for an IV pump may provide information regarding the bag content level, line obstruction, and/or the cause of the line obstruction. A feeding pump alarm may provide information regarding whether the line went dry or the pump was paused. A request for assistance may provide information regarding patient activity and basic vital signs.

In some aspects, the alarm management system 124 may determine that patient action may be used to address the alarm. The system may activate virtual agent generation module 138 and a virtual agent 118 may appear on a display. The virtual agent 118 may provide instructions to the patient 106 to perform some action. For example, the virtual agent 118 may instruct the patient to re-attach a sensor or to change position. In another example, the virtual agent 118 may instruct the patient to hold still for a specific length of time, for example, during data acquisition from the one or more sensors attached to the patient 106. In other aspects, the virtual agent 118 may instruct the patient to move to a position in which a sensor can be read. In some aspects, the virtual agent 118 may perform a level of care assessment (LOC) to determine a patient's condition. Such information can then be entered into the patient's chart.

In some aspects, an image may be analzyed in a series of stages. For example, an object may be identified in an image in a first step, the state of the object may be identified (e.g. on, off, error) in a second step, and the information conveyed by the object may be extracted in a third step. In some aspects, there may be a triggering event for monitoring. For example, if a healthcare provider enters the room, the devices may be re-evaluated to determine if the state has changed. If a device has been turned off, then device monitoring may be suspended. If a device is turned on, monitoring may be activated.

For example, an infusion pump may be identified as being on or off. If it is on, the time since it was started may be tracked. If an alarm on the infusion pump sounds, the bag attached to the pump may be identified as empty, full, or partially full. The amount of fluid in the bag relative to the time it has been running may be determined. The infusion pump may be evaluated for occlusion, flow error, air-in-line, end of infusion, near end of infusion, and/or syringe disengagement. Using image analysis, with or without additional sensor data, the system may identify the type of error and convey that information to a clinician.

In some aspects, evaluations are made dynamically depending on what object has been identified. For example, a patient's pose estimation may be analyzed more frequently than an idle device. In some aspects, the objects in a room may be evaluated collectively. That is, the state of an object in a patient's room may be evaluated in terms of other objects in a room. In other aspects, the electronic medical record may provide context. For example, if an update is sent to the electronic medical record, then the context of the devices may be analyzed relative to the record update. For example, if units of blood are being transfused, when a new bag is scanned and entered into the EMR, the system may start monitoring the IV pump.

The machine learning model for use with object identification, context generation, and input analysis, may include an artificial neural network, a decision tree, a regression algorithm, or another machine learning algorithm to determine one or more objects in the image data. The machine learning model may be trained in a variety of ways, for example, using training data including other image data including one or more objects and movement of the objects or context for the objects. Using the training data, the machine learning model may be trained to detect and/or identify objects and movement of the objects within the image data. Moreover, the machine learning model may use image data previously input into the machine learning model to continue to train the machine learning model, thus increasing the accuracy of the machine learning model. In some aspects, one or more objects, movements, or reflections identified through the machine learning model may be weighted depending on specifics related to the patient, the patient's condition, or the type of data being collected.

The machine learning model may additionally identify responses to alarms and use the responses as training data to identify, create, or suggest a response to the alarm. In some aspects, one or more actions and alarms may be weighted depending on specifics related to the patient, the patient's condition, or the type of data being collected.

Machine learning systems may take advantage of data to capture characteristics of interest having an unknown underlying probability distribution. Machine learning may be used to identify possible relations between observed variables. Machine learning may also be used to recognize complex patterns and make machine decisions based on input data. In some examples, machine learning systems may generalize from the available data to produce a useful output, such as when the amount of available data is too large to be used efficiently or practically. As applied to the present technology, machine learning may be used to learn which performance characteristics are preserved during a localization process and validate localized content when the performance characteristics are preserved.

Machine learning may be performed using a wide variety of methods or combinations of methods, such as contrastive learning, supervised learning, unsupervised learning, temporal difference learning, reinforcement learning, and so forth. Some non-limiting examples of supervised learning which may be used with the present technology include AODE (averaged one-dependence estimators), artificial neural network, back propagation, Bayesian statistics, naive bayes classifier, Bayesian network, Bayesian knowledge base, case-based reasoning, decision trees, inductive logic programming, Gaussian process regression, gene expression programming, group method of data handling (GMDH), learning automata, learning vector quantization, minimum message length (decision trees, decision graphs, etc.), lazy learning, instance-based learning, nearest neighbor algorithm, analogical modeling, probably approximately correct (PAC) learning, ripple down rules, a knowledge acquisition methodology, symbolic machine learning algorithms, subsymbolic machine learning algorithms, support vector machines, random forests, ensembles of classifiers, bootstrap aggregating (bagging), boosting (meta-algorithm), ordinal classification, regression analysis, information fuzzy networks (IFN), statistical classification, linear classifiers, fisher's linear discriminant, logistic regression, perceptron, support vector machines, quadratic classifiers, k-nearest neighbor, hidden Markov models and boosting. Some non-limiting examples of unsupervised learning which may be used with the present technology include artificial neural network, data clustering, expectation-maximization, self-organizing map, radial basis function network, vector quantization, generative topographic map, information bottleneck method, IBSEAD (distributed autonomous entity systems based interaction), association rule learning, apriori algorithm, eclat algorithm, FP-growth algorithm, hierarchical clustering, single-linkage clustering, conceptual clustering, partitional clustering, k-means algorithm, fuzzy clustering, and reinforcement learning. Some non-limiting example of temporal difference learning may include Q-learning and learning automata. Another example of machine learning includes data pre-processing. Specific details regarding any of the examples of supervised, unsupervised, temporal difference or other machine learning described in this paragraph that are generally known are also considered to be within the scope of this disclosure. Support vector machines (SVMs) and regression are a couple of specific examples of machine learning that may be used in the present technology.

In some examples, the machine learning module 128 may include access to or versions of multiple different machine learning models that may be implemented and/or trained according to the techniques described herein. For example, the machine learning model may be trained using annotated video data of patient care facilities with annotations of event data describing events visible within the video data. The machine learning module may be trained using vocal or mechanical input from the patient. The machine learning model may then be capable of receiving video data and outputting identifications and/or annotations of events contained or represented within the video data. The machine learning model may be continually updated and/or refined as additional types of events are added to the training data, for example, when a new procedure or task is added to a nurse's workflow, the training data may be updated with video data of the procedure with associated annotations. Any suitable machine learning algorithm may be implemented by the machine learning module 128. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like.

The alarm management system 124 may use one or more of the machine learning models from the machine learning module 128 to provide context for or execute routines. For example, the machine learning models may include models for object recognition, audio speech recognition, sensor data processing, person identification, and the like for use in generating alarm context in the patient room.

In some aspects, the patient 106 may be associated with a unique identifier that is displayed, included in, indicated by, and/or otherwise provided by an identifier such as an identification band. The identification may include any suitable visible symbol for encoding information, such as a barcode, quick-read (QR) code, alphanumeric string, or other visual identifier. Accordingly, the patient 106 may be identified using data from the data collection device 102 that includes a representation of the patient as well as the identifier.

The identification of patient 106 and other objects within the room may use one or more machine learning techniques for object and/or person recognition. The servers 142 or other computing devices such as a device in the patient's room, may house one or more machine learning models to perform such tasks. To aid with the identification, patients and caregivers may have visual identifiers that may be visible to the camera and/or be enrolled with a facial recognition system. For example, data collection device 102 may be used to enroll patients 106 and/or caregivers 146. The data collection device 102 may capture image data of the caregiver 146 and associated credentials and/or identifiers included in an ID. The ID may be associated with a caregiver profile stored in association with the servers 142 in the system such that when the caregiver 146 is enrolled, they may be readily identified by the computing system for various purposes, with or without the ID.

The identification and tracking of patients and individuals within the patient room of the care facility enables the system to track individuals within video and other sensor data and, using machine learning models, identify events related to the patient or within the patient room. Additionally, the identification systems may be used for identifying objects, such as medical devices, treatment devices, medications, and other objects within the room of the patient.

The data collection device 102 may be used to capture image and/or video data that may be analyzed to determine events occurring within the room. In some examples, sensors such as sensor 108, and/or sensors integrated with medical devices such as infusion pump 110 may output data that may be used, in conjunction with the data from the data collection device 102, to determine events. The events may represent an action taken by a caregiver 146, the patient 106, a visitor, or other individual. The alarm management system 124 may use the data to generate context or conditions for the event including an alarm indication using context generation module 140. The context may be compared to one or more rules by the decision module 136 or may be used as a prompt for generative AI and the alarm management system 124 may execute the action provided by the decision module 136 to address the alarm. Such execution may initiate caregiver, patient, and/or virtual agent action. Once the action has been completed, the alarm management system 124 may identify and take one or more additional actions.

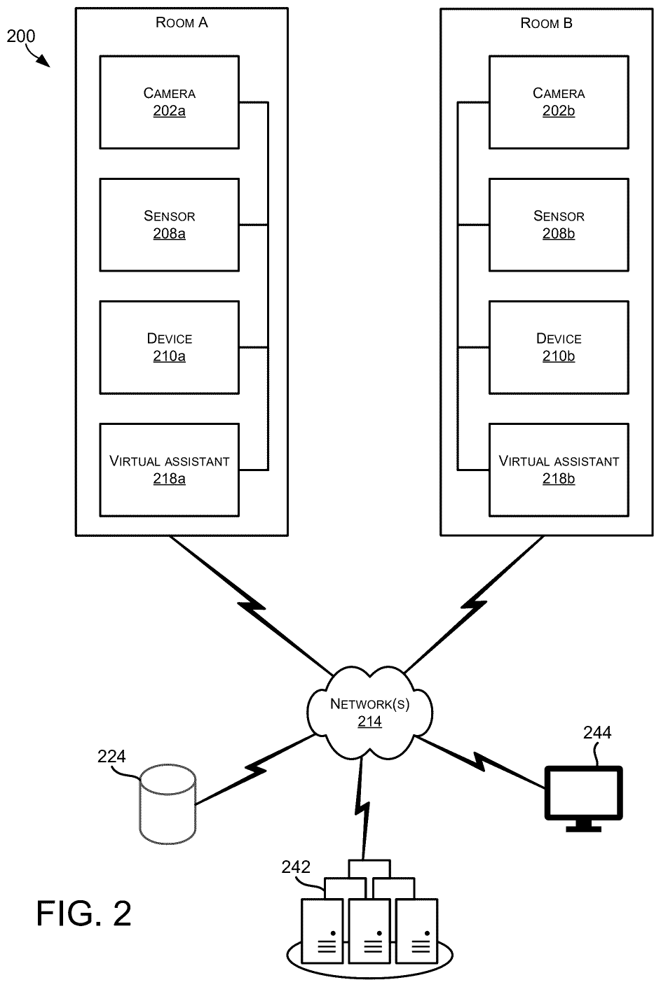

FIG. 2 illustrates a system architecture 200 for monitoring patients in multiple rooms or room areas (for example portions of single room where each portion contains a single patient), according to at least one example. The system architecture 200 includes components similar or identical to those of FIG. 1, such as the alarm management system 224, computer system and databases 242, display 244, respective data collection devices such as imaging device 202a and imaging device 202b, respective sensor 208a and sensor 208b, respective device(s) 210a and medical device 210b, and respective virtual agent 218a and virtual agent 218b which may correspond to the alarm management system 124, servers 142, display device 144, data collection device 102, sensor 108, medical device(s) such as infusion pump 110, and virtual agent 118 of FIG. 1.

In the system architecture of FIG. 2, there are two rooms, Room A and Room B. Each room includes a camera or other imaging device, at least one sensor, a medical device, and a virtual agent. One or more of the various devices in each room may communicate with the network 214 to exchange information with the alarm management system 224, the databases 242 on servers, and receive and present information to one or more display devices similar to display 244. Images and other data acquired from the medical devices within each room may be sent to alarm management system 224 for further processing or, alternatively, such messages may be forwarded directly to one or more other computer devices that are in communication with network 214, such as, but not limited to, an electronic medical records (EMR) computer device, a work flow management computer device, a caregiver alerts computer device, an admissions, discharge, and transfer (ADT) computer device, or any other computer device in communication with network 214. Computer devices provide the software intelligence for processing the images, depth sensor data, and/or voice data recorded by cameras, and the precise physical location of this intelligence can vary in a wide variety of different manners, from embodiments in which all the intelligence is centrally located to other embodiments wherein multiple computing structures are included and the intelligence is physically distributed throughout the caregiving facility.

The databases 242 on servers may contain information that is useful for one or more of the algorithms carried out by system architecture 200. This information may include photographic and/or other physical characteristic information of all of the current clinicians and/or staff of the patient care facility so that the object identification module of the alarm maanagmeent system 224 can compare this information to the signals detected by imaging devices 202a and 202b to identify if a person is a hospital employee and/or who the employee is. This information may also include photographic and/or other physical data of the current patients within the patient care facility so that patients can be recognized. The information within databases 242 on servers may also include data that is specific to individual rooms within the facility, such as the layout of the room, where and what objects are positioned within the room, the dimensions of the room, the location of room doors, and other useful information. The database may also include identifying information for identifying objects and assets, such as equipment used within the room. Such identifying information may include information about the shape, size, colors, and/or states of objects that the computing device is designed to detect.

The system architecture 200 is configured to detect people and other objects that appear in the images detected by imaging devices 202a and 202b. The detection of such people can be carried out in known manners, as would be known to one of ordinary skill in the art. In general, imaging devices 202a and 202b may be positioned to record image information useful for any one or more of the following purposes: ensuring proper patient care protocols are followed; identifying the type of behavior of a patient or the patient's condition; detecting alarms, and/or providing context for an alarm.

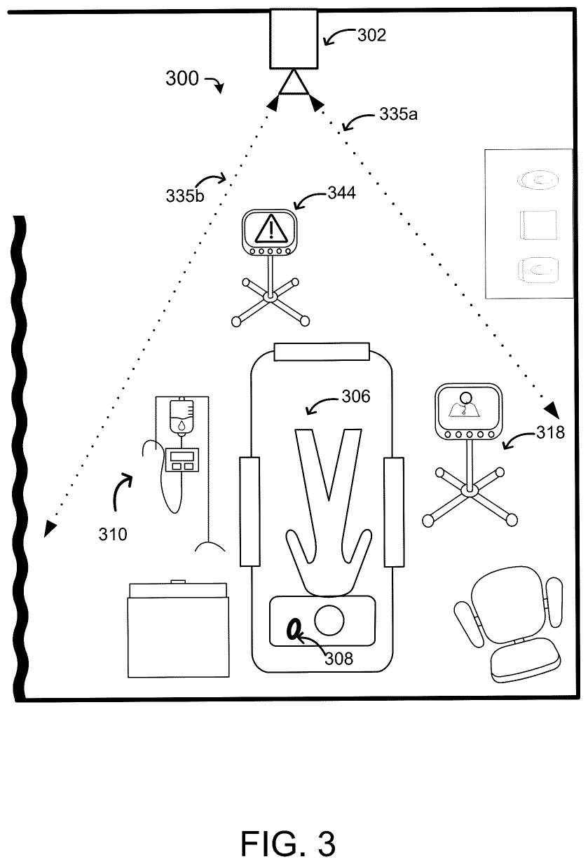

FIG. 3 illustrates a top-view of an environment 300 for a patient management system. For example, the environment 300 is illustrated as a hospital room housing a patient 306. However, this application anticipates the environment 300 being any healthcare setting in which a patient may be observed, such as an operating room, an outpatient facility, a clinical lab, to name a few non-limiting examples. In some aspects, environment 300 may be a home environment. The environment 300 may include a data collection device 302 similar to data collection device 102, a patient 306 similar to patient 106, a sensor 308 similar to sensor 108, a medical device such as infusion pump 310 similar to infusion pump 110, display device 344 similar to display device 144, and a virtual agent 318 similar to virtual agent 118 as described above with respect to FIG. 1.

The data collection device 302 is an imaging device, it may have a field of view as illustrated by dotted lines 335a and 335b. Within the field of view, there is a set of objects. As shown in FIG. 3, exemplary objects may include a patient 306, a sensor 308 on the patient, one or more medical devices exemplified by infusion pump 310, one or more computer or display devices 344, and a virtual agent 318. Additional displays, people, and devices may be included in other configurations. In some aspects the imaging device may pan or zoom, thus the field of view encompassed by 3356a and 335b may change depending on the position or setting of the camera.

The alarm management system 124 may analyze one or more images or frames received from the imaging device to identify the objects and the state of the objects within the room. For example, the system may identify that the virtual agent 318 and the infusion pump 310 are adjacent to the patient 306. As the infusion pump 310 is not attached to the patient, the system may determine whether the infusion pump 310 should be attached to the patient. For example, the system may analyze the electronic medical record (EMR), compare the current image to an image taken at a prior point or time, or identify whether the infusion pump 310 should be turned on or off. In some aspects, the virtual agent 318 may ask the patient whether the infusion pump should be attached. In the event that the system determines that the infusion pump should be connected to the patient, an alarm may sound and a notification may be sent to a caregiver. If the alarm management system 124 determines that the infusion pump is correctly detached from the patient, the system may disregard the infusion pump until there is a change in state. For example, if the alarm management system 124 determines at a later point in time that the infusion pump has been turned on.

In some aspects, an alarm related to sensor 308 may sound. Images acquired at the time of the alarm may be analyzed using machine learning and the system may determine that the sensor 308 is on a pillow instead of a patient 306. The alarm management system 124 may access the decision module to determine what action to take if a sensor 308 is on a pillow or otherwise detached from patient 306. The action may then be passed onto the virtual agent 318 or a clinician. For example, the alarm management system 124 may activate the virtual agent 318 and the virtual agent may instruct the patient to reattach the sensor 308. Reattaching the sensor 308 to the patient 106 may allow the alarm management system to re-set the alarm without necessitating clinician intervention. In some aspects, the alarm management system 124 may identify that the sensor has been detached before an alarm has sounded. That is, through continuous or intermittent monitoring, the system may identify that a sensor is detached, determine that a sensor should be attached, and activate a virtual agent to instruct the patient to attach the sensor prior to an alarm sounding.

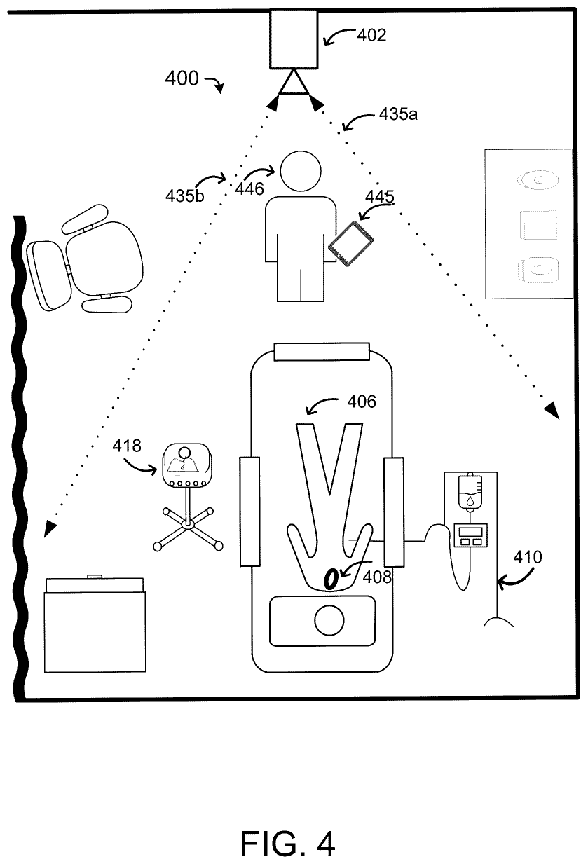

FIG. 4 illustrates a top-view of an environment 400 for a patient management system. For example, the environment 400 is illustrated as a hospital room housing a patient 406. However, this application anticipates the environment 400 being any healthcare setting in which a patient may be observed, such as an operating room, an outpatient facility, a clinical lab, to name a few non-limiting examples. In some aspects, environment 400 may be a home environment. The patient management system environment 400 may include an camera for use as a data collection device 402 similar to data collection device 102, a patient 406 similar to patient 106, a sensor 408 similar to sensor 108, a medical device such as infusion pump 410 similar to infusion pump 110, a virtual agent 418 similar to virtual agent 118, and a caregiver 446 similar to caregiver 146 as described above with respect to FIG. 1.

The imaging device may have a field of view illustrated by dotted lines 435a and 435b. Within the field of view, there is a set of objects. As shown in FIG. 4, exemplary objects may include a patient 406, a sensor 408 on the patient, one or more medical devices as represented by the infusion pump 410, a virtual agent 418 on a display, and a caregiver 446 with a tablet or other device 445. In some aspects, patient related information may be displayed on a separate monitor similar to display device 144. Additional displays, people, and devices may be included in other configurations. In some aspects, the imaging device 402 may pan or zoom, thus the field of view encompassed by dotted lines 435a and 435b may change depending on the position or setting of the imaging device such as a camera.

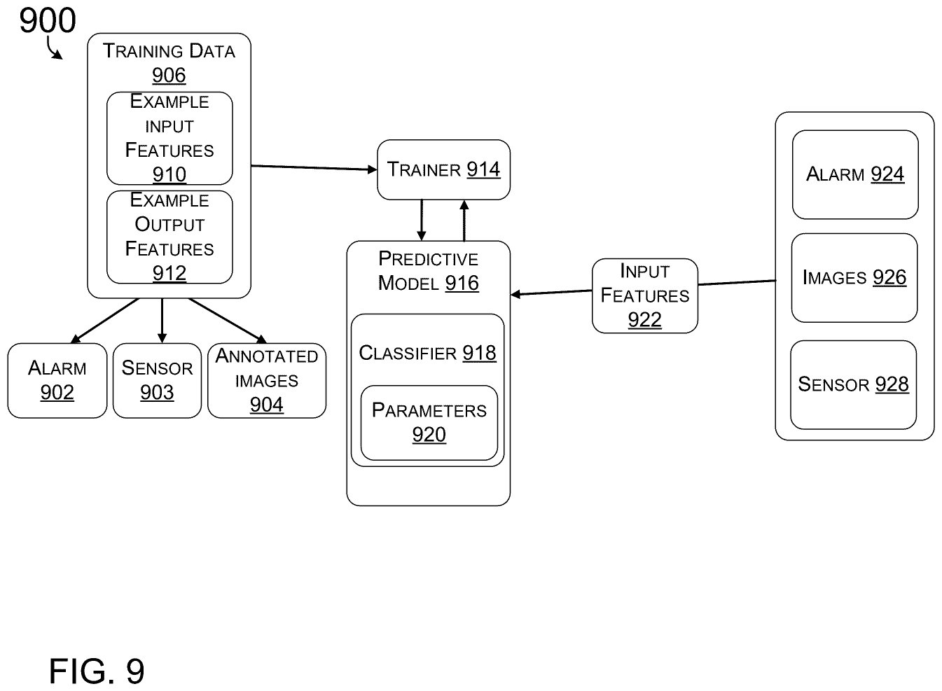

For example, the image may be analyzed using, for example, machine learning, and the alarm management system may identify that the virtual agent 418 and the infusion pump 410 are adjacent to the patient 406. The alarm management system 124 may additionally identify that the infusion pump 410 is attached to the patient. If an alarm on the infusion pump 410 sounds, images acquired by the data collection device 402 prior to or during the alarm may be analyzed to determine a possible source of the alarm. In some aspects, the alarm management system 124 may compare current images to previous images in which the alarm was not sounding to identify differences between the current state of the infusion pump 410 and a previous state of the infusion pump 410. As shown in FIG. 4, it appears that the IV line from the infusion pump 410 is under the arm of the patient 406. The alarm management system may instruct the virtual agent to interact with patient 406 and instruct the patient to change position. If changing position addresses the issue, the alarm management system may re-set the alarm. In other aspects, the identification of an issue may take place in multiple steps. For example, an image may be acquired by data collection device 402 with a field of view as shown by dotted lines 435a and 435b. A first machine learning model may be used to identify events within the room, that is, the first machine learning model may identify that an alarm has sounded. A second machine learning model may be used for object detection of relevant objects associated with the events (e.g., equipment used or interacted with). For example, upon detection of an alarm, the alarm management system 124 may initiate image acquisition via imaging device 402. The second machine learning model may be used to parse the images to detect the objects within the field of view using, for example, object identification module 134 and identify the objects such as the caregiver 446, the device 445, the bed, the patient 406, the display device 445, the infusion pump 410, and the desk. A third machine learning model may determine the identity of the individuals within the room, for example, the identity of the patient 406 and the identity of the caregiver 446. A fourth machine learning model may identify the context for the alarm based on the event, object, and people in the room. For example, the fourth machine learning model may identify why the alarm for the infusion pump 410 has sounded. In some aspects, the fourth model may be trained, for example using the system of FIG. 9. Based on the cause of the alarm and the people or other objects in the room, the alarm management system 124 may initiate the virtual agent to suggest to the patient an action for the patient to take. As shown in FIG. 4, the action may be instructing the patient to move to free the line from the infusion pump 410.

In some aspects, one or more of the machine learning models may continuously acquire and analyze information. In some aspects, the models may include information acquired at previous time points. For example, if particular alarms are triggered, the system may review records for causal events such as actions that have taken place within a threshold time period, or expected actions that did not take place within a threshold time period. Such actions may include clinician or patient actions such as movement, medication administration, and the like.

FIG. 5 provides an example of the types of information that may be provided to a caregiver on a display 502 and used to provide context for the caregiver. For example, as shown in FIG. 5, on the display 502, there may be patient information included in subscreen 512. The patient's vitals may be displayed at 504. Additional sensors may provide readings at 506. In some aspects, device specific information may be shown at 508 and 510. The display 502 may appear on a monitor such as display device 144, on a handheld device such as device 445, or some other local or remote display that can be viewed by a caregiver. When an alarm sounds, the display 502 may provide additional context for the alarm. For example, the display 502 may indicate which device or what condition triggered the alarm. In some aspects, display 502 may provide an image of the device causing the alarm. Using the display 502, the caregiver may provide instructions to the alarm management system 124. For example, if, using the virtual agent, the alarm management system 124 was unable to resolve the alarm, the alarm management system may send an image of the device to the caregiver and the caregiver may provide additional instructions via the virtual agent or verbal interaction with the patient to resolve the alarm.

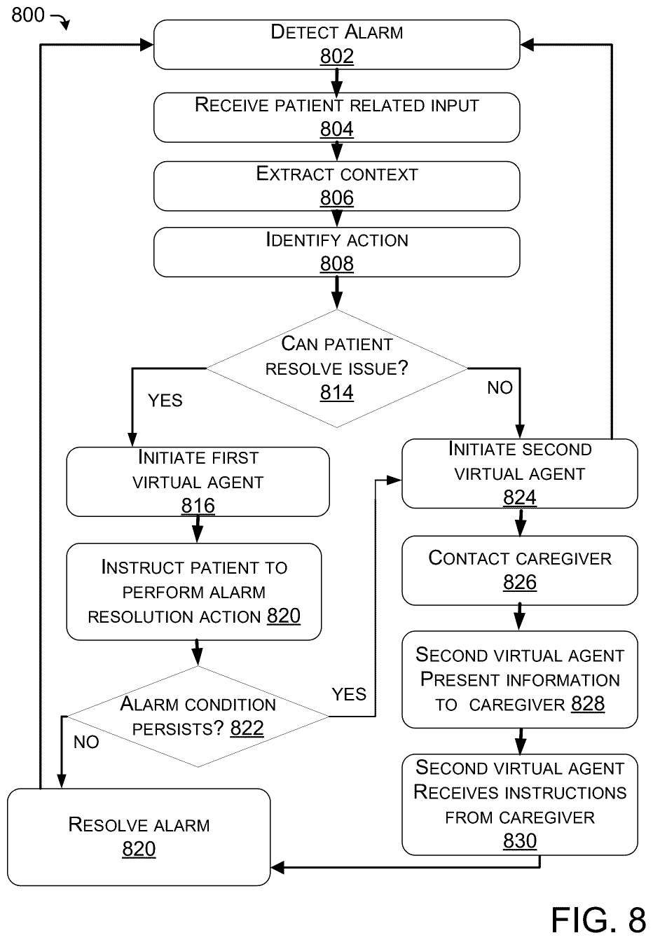

For example, a device may send a signal that a bag of fluid is empty or that a fluid has ceased to flow using icon 508. In other aspects, such as if a device is not networked, the system such as alarm management system environment 100 may have analyzed a room such as the room shown in FIG. 3 or FIG. 4 and determined the level of fluid in the IV bag such of infusion machine such as infusion pump 110, infusion pump 310, or infusion pump 410. Icon 510 may provide information regarding the type of fluids, the amount, and the frequency of the delivery. Thus, a care provider has access to information from a variety of sources and if an alarm sounds, the information may be evaluated in view of other devices and/or objects within the room, providing additional context as to the state of the patient and allowing the caregiver to provide additional instructions to the alarm management system 124 including resetting the alarm. In some aspects, such displays may be used as part of an interaction of a caregiver with a second virtual agent as described in further detail with regard to FIG. 8.

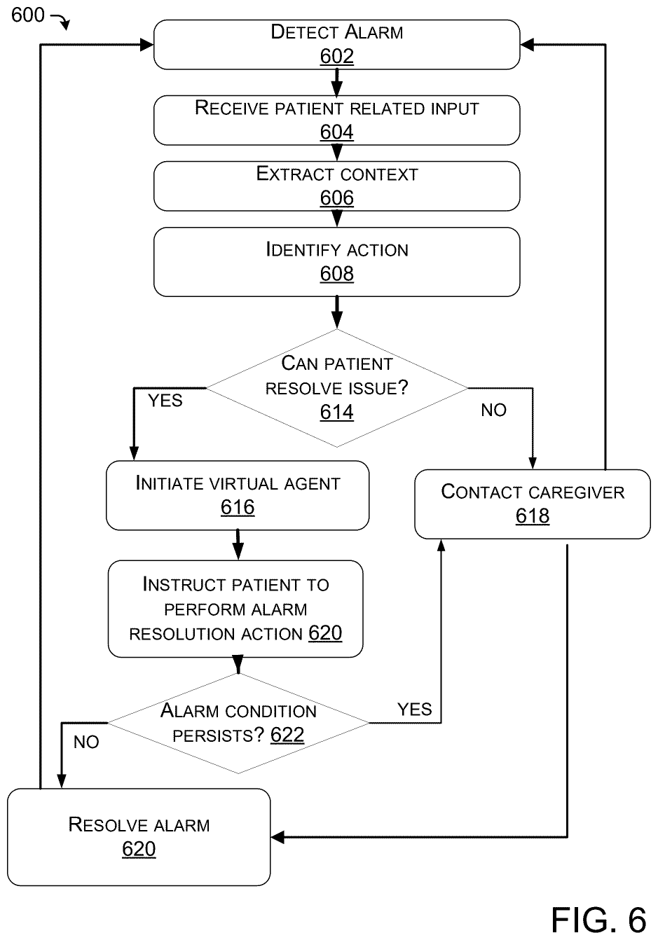

FIG. 6 is an embodiment of alarm management system 600. The system detects an alarm in a patient's room at 602. The system then receives patient related input at 604. Such patient related input may be from a variety of sources such as an image, a sensor, verbal utterings, a button being pushed, and the like. The machine learning system such as machine learning module 128 extracts context from the patient related input at 606 alone or in combination with other information from other devices within or images of the room, determines a next action at 608, and using the extracted context determines the appropriate rule. In some aspects, the machine learning model may be trained using training data including annotated video data of patient care with annotations of event data. In some examples, a first machine learning model may be used to identify events within the room, a second machine learning model may be used for object detection of relevant objects associated with the events (e.g., equipment used or interacted with), a third machine learning model may prioritize the alarm, a fourth machine learning model may identify individuals within the room, and a fifth machine learning model may provide context for an alarm based on the event, object, and people in the room.

For example, a patient may request assistance and the requested assistance may indicate the nature of the alarm such as when a patient tries to get out of bed but is not allowed to get out of bed independently. If devices are networked, for example wirelessly connected to each other using for example a mesh network, the system may use information transmitted by the device to identify the device and the condition that prompted the alarm. If the device is not networked or the patient has not relayed the cause of the alarm, images may be analyzed using machine learning to provide more information. Analysis of an image may identify a cluster of objects associated with a patient. For example, there may be a plurality of devices/sensors within a room such as a pulse oximeter, a blood pressure cuff, an infusion pump, an oxygen mask, bed exit alarm, patient monitor, and the like. The system may identify the state of each object, that is, is the device on or off, the source of the alarm, the identity of the patient, the identity of anyone proximate to the patient, and the like. The system may then compare the current state of each object to the state of the object in a previous frame to determine if the state has changed. For example, if there is an oxygen mask in the room, but there is no information in the record of the patient being on oxygen, and the previous frames indicate that the oxygen system has not been used, the system may determine that the state of the oxygen mask is “off” and the state of the oxygen mask should not be used to provide context to an alarm. If the record indicates that the patient is on oxygen and in a previous frame the system determines that a patient is wearing an oxygen mask, the state of the oxygen or oxygen mask may be used to provide context for an alarm. For example, if the patient has not been on oxygen, the fact that the patient is currently not on oxygen is not relevant to the context of the alarm. If the patient has been on oygen and the oxygen mask is no longer attached, the object is relevant to the determination of context, as the state of the object has changed. The system adds the state to a collection of object states and uses machine learning or generative AI to extract the context of the alarm at 606 from the collection of relevant objects.

The system then determines a next step at 608 and determines if the patient can resolve the cause of the alarm 614. If there is no action that a patient could take that would resolve the alarm, a caregiver is notified at 618. If there is an action a patient can take to respond to the alarm, the system executes the action. In some aspects, execution may include activating a virtual agent at 616. The virtual agent may then interact with the patient in an attempt to address the detected alarm at 620. For example, if the extracted context indicates that a sensor has become detached, the virtual agent may request that the patient re-attach the sensor. If the extracted context indicates that a line has become kinked, impeding flow, the virtual agent may instruct the patient to change position. If the extracted context indicates that measurements can not be acquired because of the patient's position or movemements, the patient may be asked to change position or stay still. If, after interaction with the virtual agent, the alarm condition persists at 622, a caregiver may be contacted. If the patient interaction resolves the issue generating the alarm at 620, the system may reset the alarm, decreasing the need for caregiver intervention.

If at 618 it is necessary to notify the caregiver, a second virtual agent may be created as shown in more detail in FIG. 8. The descion module can determine the role that the caregiver could execute to resolve the alarm. For example the caregiver may be notified that a caregiver needs to re-attach a patch, switch out an IV, or the like. That information will be included in an agent along with the other context before a decision was made to contact the nurse. In some aspects, the caregiver may ask the virtual agent to provide additional information for example, what was the patient doing prior to the alarm going off. In some aspects, this may be, hands free communication with the nurse. In some aspects, the nurse and/or the virtual agent may be able to take action remotely. For example, if the patient is asleep, but there is a fault with a sensor or it is attached incorrectly, the virtual agent or caregiver may instruct the system to switch to a second lead or an alternate power source. In other aspects, the caregiver may need to perform a manual manipulation and will enter the patient's room. The virtual assistant(s) may prioritize the alarm based on the urgency and risk level and assumes that the nurse otherwise occupied at the time of the alarm. Such a determination may be made using a rule based system or machine learning as described in further detail herein.

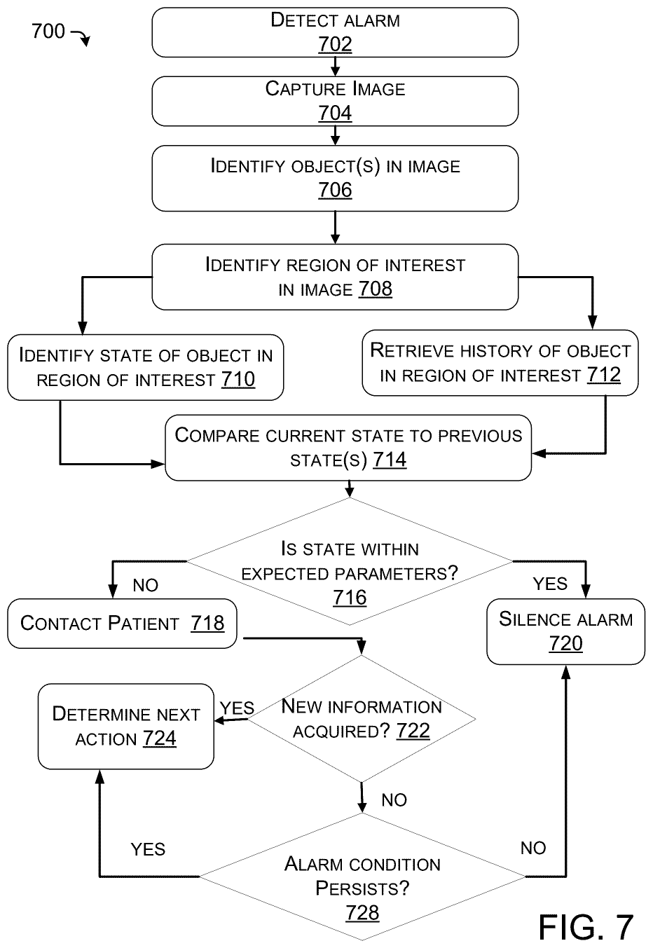

As shown in FIG. 7, the system 700 may detect an alarm at 702. After the alarm is detected, the system may capture an image of the device that triggered the alarm or an image of the patient room as a whole at 704. The system may then analyze the image(s) using machine learning to identify the object(s) in the image at 706 and a region of interest related to the alarm at 708. For example, if a first object is identified as a patient, the system may identify the objects that are connected to the patient and then identify the state of the object(s) connected to the patient, ignoring the objects that are not connected to the patient, that is, the object(s) connected to the patient are in a region of interest. Once the relevant object(s) are identified, the system may identify the state of the object(s) at 710, that is, whether the object is on/off, functioning as normal, and any readings displayed or generated by a device. The system may also retrieve the history of the object at 712. Such a history may include EMR notations, medication history, medication orders, medication administration, previous settings for a device, previous states of the device, or patient specific requirements. Such a history may include the severity of the patient's illness, various levels of monitoring the patient, or the prioritization of one or more alarms based on the condition or specific patient. The system may then compare the current state of the object to prior information at 714. The system may then determine if the state of the object is within the expected or allowed parameters at 716. If the object is within the expected or allowed parameters, the system may silence the alarm at 720. In the event that the object is not operating within expected parameters, the system may contact the patient 718. The virtual agent may then ask the patient a series of questions or may ask the patient to perform one or more actions. Based on the information received from the patient at 722 including a change in position, the system may determine a next action at 724. If no information is obtained, or if the condition persists at 728 despite the interaction with the patient, the the next action may be determined at 724. Such a next action may be one or more of a variety of different possiblities including further interaction with the patient and/or contact with a caregiver.

FIG. 8 is an embodiment of alarm management system 800 similar to the embodiment of alarm management system 600 with the addition of a second virtual agent at 824. In the system 800, the system detects an alarm associated with a patient at 802. The system then receives patient related input at 804. Such patient related input may be from a variety of sources such as an image, a sensor, verbal utterings, a button being pushed, and the like. The machine learning system such as machine learning module 128 extracts context from the patient related input at 806 and in combination with other context provided by the one or more sensors, images or devices within the patient room, determines a next action at 808, and using the extracted context determines the appropriate rule. In some aspects, the machine learning model may be trained using training data including annotated video data of patient care with annotations of event data. In some examples, a first machine learning model may be used to identify events within the room, a second machine learning model may be used for object detection of relevant objects associated with the events (e.g., equipment used or interacted with), a third machine learning model may prioritize the alarm, a fourth machine learning model may identify individuals within the room, and a fifth machine learning model may provide context for an alarm based on the event, object, and people in the room. In some aspects, the system may prioritize an alarm either independently or based on instructions of the caregiver at 830.

For example, a patient may want to get out of their bed and an alarm may sound, for example when a patient tries to get out of bed but is not allowed to get out of bed independently. As the patient is not allowed to get out of bed independently, the first virtual agent at 816 may be initiated and instructions may be sent to the patient at 820 indicating that the patient is not allowed to be out of bed and the patient should not attempt to do so. If the alarm condition persists at 822, that is, the patient continues to try and get out of bed, the system may initiate a second virtual agent at 824. The second virtual agent may contact the caregiver at 826 and relay the issue and additional information to the caregiver at 828. Such additional information may include the information shown at FIG. 5 or other relevant details. In some aspects, the second virtual agent may receive instructions from the caregiver at 830. Such instructions may include actions for the system to take such as locking the bed rails, or vocal notification to the patient that the caregiver is on the way to assist them.

If a device is networked, that is, the devices in the room are connected to each other or to an exterior device, the system may use information transmitted by the device to identify the device and the condition that prompted the alarm. If the device is not networked or the patient has not relayed the cause of the alarm, images may be analyzed using machine learning to provide more information. Analysis of an image may identify a cluster of objects associated with a patient. For example, there may be a plurality of devices/sensors within a room such as a pulse oximeter, a blood pressure cuff, an infusion pump, an oxygen mask, bed exit alarm, patient monitor, and the like. The system may identify the state of each object, that is, is the device on or off, the source of the alarm, the identity of the patient, the identity of anyone proximate to the patient, and the like. The system may then compare the current state of each object to the state of the object in a previous frame to determine if the state has changed. For example, if there is an oxygen mask in the room, but there is no information in the record of the patient being on oxygen, and the previous frames indicate that the oxygen system has not been used, the system may determine that the state of the oxygen mask is “off” and the state of the oxygen mask should not be used to provide context to an alarm for the caregiver. If the record indicates that the patient is on oxygen and in a previous frame the system determines that a patient is wearing an oxygen mask, the state of the oxygen or oxygen mask may be used to provide context for an alarm. For example, if the patient has not been on oxygen, the fact that the patient is currently not on oxygen is not relevant to the context of the alarm. If the object is relevant to the determination of context, or the state of the object has changed, the system adds the state to a collection of object states and uses machine learning or generative AI to extract the context of the alarm at 806 from the collection of relevant objects and determine if a patient can resolve the issue at 814.

If there is an action a patient can take to respond to the alarm, the system proceeds without notifying a caregiver ins. In some aspects, execution may include activating a virtual agent at 816. The virtual agent may then interact with the patient in an attempt to address the detected alarm at 820. For example, if the extracted context indicates that a sensor has become detached, the virtual agent may request that the patient re-attach the sensor. If the extracted context indicates that a line has become kinked, impeding flow, the virtual agent may instruct the patient to change position. If the extracted context indicates that measurements can not be acquired because of the patient's position or movements, the patient may be asked to change position or stay still. If, after interaction with the virtual agent, the alarm condition persists at 822, a caregiver may be contacted. If the patient interaction resolves the issue generating the alarm at 820, the system may reset the alarm, decreasing the need for caregiver intervention.

If at 822 it is necessary to notify the caregiver, a second virtual agent may be created. The decision module can determine the role that the caregiver could execute to resolve the alarm. For example, the caregiver may be notified that a caregiver needs to re-attach a patch, switch out an IV, or the like. In other aspects, there may be actions that the caregiver could take remotely. For example, if the IV pump is finished and a second IV is not needed, the caregiver could instruct the system to turn off the alarm. If there is an issue with a lead for an electrode, the care giver could instruct the system to use an alternate lead to continue the information collection. If an outlet fails, the caregiver could instruct the system to use backup generation. In some aspects, after reviewing the information presented by the second virtual agent, the caregiver may determine that they need to attend the patient in person. In some aspects, the second virtual agent may triage the alarm, determining the priority and urgency of the alarm and convenying that information to the caregiver, assisting the caregiver in the allocation of resources based on the condition of the patient determined by the alarm management system.

FIG. 9 illustrates an example environment 900 for training and utilizing a predictive model 916 to provide context for object(s) within a patient room. The predictive model 816, for instance, is the predictive model for consequences of an alarm or actions to take when an alarm sounds. In various implementations, the predictive model 916 includes a classifier 918, which may include one or more machine learning (ML) models. A trainer 914, for instance, is configured to optimize various parameters 920 of the classifier 918 based on training data 906.

The training data 906 includes example alarm states 902, sensor data 903, or example annotated images 904 as example input features 910. The rules to be executed based on the example alarm states 902 and example annotated images 904 may form example output features 912. The example alarm states 902, in various cases, is obtained using exemplary sensor readings, exemplary transmitted information from networked devices and/or exemplary information extracted from images. The examples may additionally include annotated images 804 which may identify the objects and/or the state of the objects in a patient environment. The example output features 912 may include categorizations of various alarms from devices. Categorization may be based on the sound of the alarm, the frequency of the alarm, the type of machine that sounds the alarm, the general status of the patient and the like.

The classifier 919 includes one or more model types. For instance, the classifier 918 may include an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the classifier 918 is defined according to the parameters 920. For example, the parameters 920 may include weights, thresholds, filters, kernels, or other data objects that are utilized to perform operations of the classifier 918.

In some implementations, the classifier 918 includes a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 920 may include k (e.g., the number of neighbors compared to the new data point), the threshold distance, and so on.

In various cases, the classifier 918 includes a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 920.

In some cases, the classifier 918 includes a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 920, for example, include a threshold proximity within which a new data point is classified within a cluster, a density of points used to define a cluster, and the like.

In various examples, the classifier 918 includes a principal component analysis model. In various implementations, a principal component analysis defines a collection of principal components of unit vectors within a coordinate space based on a data set (e.g., training data). The model, for example, is an orthogonal linear transformation of the data set. Various weights of the model, for example, are included in the parameter(s) 920.

The classifier 918 in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 920.

The classifier 918, for example, includes a random forest. The random forest, for instance, includes multiple decision trees that classify data in an ensemble fashion. In various implementations, the decision trees are defined by the parameter(s) 920.

In various implementations of the present disclosure, the trainer 914 is configured to optimize the parameters 920 based on the training data 906. For example, the trainer 914 may input first example features (corresponding to alarm state 902) and/or second example features (corresponding to annotated images 904) into the predictive model 916, and may receive a predicted category. The trainer 914 may compute a loss (e.g., determine a discrepancy) between a first example category (corresponding to a first alarm) among the example output features 912 and the predicted category. Further, the trainer 914 may alter the parameters 920 in order to minimize the loss. In various cases, the trainer 914 optimizes the parameters 920 iteratively based on the entire set of the training data 906.

In various implementations, the optimization of the parameters 920 enables the predictive model 916 to identify attributes of the alarm 924 and annotated images 926 that are correlated to or otherwise associated with the example output features 912. The predictive model 816 may therefore classify context based on the alarm sounded and the images acquired on the patient environment based on the variety of states that trigger an alarm recognizing or otherwise identifying the rule related attributes.

Once the parameters 920 are optimized, the predictive model 916 may be ready to classify a new set of data. For example, the predictive model 916 may receive input data including features 922 of alarm 924, images 926, and sensors 928. The features 922, for instance, may include one or more of the predictive attributes. The predictive model 916 may perform various operations on the input data based on the trained classifier 918 and the optimized parameters 920. In various cases, the predictive model 916 outputs output data including one or more category indicators based on the input features 910. Although FIG. 9 is primarily described as referring to supervised learning, implementations are not so limited. While FIG. 9 provides an exemplary model, in some aspects other types of machine learning may be used including, for example, a large language model.

FIG. 10 illustrates an example system generally at 1000 that includes a computing device 1002 that is representative of one or more computing systems and/or devices that may implement the various techniques described herein. This is illustrated through the inclusion of the alarm management system 124. For example, the system 1000 may be configured to execute the processes of FIGS. 6 to 9. The computing device 1002 may be, for example, a server of a service provider, a device associated with a client (e.g., a client device), an on-chip system, and/or any other suitable computing device or computing system.

The computing device 1002 as illustrated includes a processing system 1004, one or more computer-readable media 1006, and one or more I/O interface 1008 that are communicatively coupled, one to another. In some embodiments, the processor(s) of the processing system includes a Central Processing Unit (CPU), a Graphics Processing Unit (GPU), or both CPU and GPU, or other processing unit or component known in the art. Although not shown, the computing device 1002 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1004 is representative of the functionality used to perform one or more operations using hardware. Accordingly, the processing system 1004 is illustrated as including hardware element 1010 that may be configured as processors, functional blocks, and so forth. This may include implementation in hardware as an application specific integrated circuit or other logic device formed using one or more semiconductors. The hardware elements 1010 are not limited by the materials from which they are formed or the processing mechanisms employed therein. For example, processors may be comprised of semiconductor(s) and/or transistors (e.g., electronic integrated circuits (ICS)). In such a context, processor-executable instructions may be electronically executable instructions.

The computer-readable media 1006 is illustrated as including memory/storage component 1012. The memory/storage component 1012 stores instructions that, when executed by the processing system 1004, causes the processing system 1004 to perform various operations. In various examples, the memory/storage component 1012 stores methods, threads, processes, applications, objects, modules, any other sort of executable instruction, or a combination thereof. In some cases, the memory 1012 stores files, databases, or a combination thereof. The storage component/memory 1012 represents memory/storage capacity associated with one or more computer-readable media. The memory/storage component 1012 may include volatile media (such as random access memory (RAM)) and/or nonvolatile media (such as read-only memory (ROM), Flash memory, optical disks, magnetic disks, and so forth). The memory/storage component 1012 may include fixed media (e.g., RAM, ROM, a fixed hard drive, and so on) as well as removable media (e.g., Flash memory, a removable hard drive, an optical disc, and so forth). The computer-readable media 1006 may be configured in a variety of other ways as further described below.

I/O interface 1008 (Input/Output interface) is representative of functionality to allow a user to enter commands and information to computing device 1002, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1002 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” “logic,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules, techniques, and flowcharts may be stored on and/or transmitted across some form of computer-readable media. The computer-readable media may include a variety of media that may be accessed by the computing device 1002. By way of example, and not limitation, computer-readable media may include “computer-readable storage media” and “computer-readable transmission media.”

“Computer-readable storage media” may refer to media and/or devices that enable persistent and/or non-transitory storage of information in contrast to mere signal transmission, carrier waves, or signals per se. Thus, computer-readable storage media refers to non-signal-bearing media. The computer-readable storage media includes hardware such as volatile and non-volatile, removable and non-removable media, and/or storage devices implemented in a method or technology suitable for storage of information such as computer-readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable transmission media” may refer to a medium that is configured to transmit instructions to the hardware of the computing device 1002, such as via a network. Computer-readable transmission media typically may transmit computer-readable instructions, data structures, program modules, or other data in a modulated data signal, such as carrier waves, data signals, or other transport mechanisms. Computer-readable transmission media also include any information delivery media. The term “modulated data signal” means a signal that has one or more of its characteristics set or changed in such a manner as to encode information in the signal. By way of example, and not limitation, computer-readable transmission media include wired media such as a wired network or direct-wired connection, and wireless media such as acoustic, radio frequency (RF), infrared, and other wireless media.

As previously described, hardware elements 1010 and computer-readable media 1006 are representative of modules, programmable device logic, and/or device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1010. The computing device 1002 may be configured to implement particular instructions and/or functions corresponding to the software and/or hardware modules. Accordingly, implementation of a module that is executable by the computing device 1002 as software may be achieved at least partially in hardware, e.g., through the use of computer-readable storage media and/or hardware elements 1010 of the processing system 1004. The instructions and/or functions may be executable/operable by one or more articles of manufacture (for example, one or more computing devices 1002 and/or processing systems 1004) to implement techniques, modules, and examples described herein.

The techniques described herein may be supported by various configurations of the computing device 1002 and are not limited to the specific examples of the techniques described herein. This functionality may also be implemented all or in part through the use of a distributed system, such as over a “cloud” 1014 via a platform 1016 as described below.

The cloud 1014 includes and/or is representative of a platform 1016 for resources 1018. Platform 1016 abstracts the underlying functionality of hardware (e.g., servers) and software resources of the cloud 1014. The resources 1018 may include applications and/or data that can be utilized while computer processing is executed on servers that are remote from the computing device 1002. Resources 1018 can also include services provided over the Internet and/or through a subscriber network, such as a cellular or Wi-Fi network.

Platform 1016 may abstract resources and functions to connect the computing device 1002 with other computing devices. The platform 1016 may also be scalable to provide a corresponding level of scale to encountered demand for the resources 1018 that are implemented via the platform 1016. Accordingly, in an interconnected device embodiment, implementation of functionality described herein may be distributed throughout multiple devices of the system 1000. For example, the functionality may be implemented in part on the computing device 1002 as well as via the platform 1016 which may represent a cloud computing environment.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as examples only, with a true scope and spirit of the present disclosure being indicated by the following claims.

### EXAMPLE CLAUSES

1. A method, including: receiving, by a computing device, an indication of an alarm, the alarm being generated by a first device associated with a patient; receiving, by the computing device, image data of a patient area, wherein the first device is at least partly disposed in the patient area; determining, by the computing device, and based on the image data, a cause of the alarm; determining, by the computing device and based on the cause of the alarm, an action required to resolve the alarm, wherein the action includes at least one of obtaining patient information or requesting that the patient perform a task; generating, by the computing device, a virtual agent configured to present an indication of the action; causing, by the computing device, the indication of the action to be presented to the patient via the virtual agent; and receiving, by the computing device, an input from the patient responsive to the indication presented by the virtual agent.

2. The method of clause 1, wherein the patient area includes the patient and a plurality of devices, the method further including determining, by the computing device and based on the image data, an identity of the first device in the plurality of devices prior to determining the cause of the alarm.

3. The method of clause 1 or 3, wherein the computing device generates the virtual agent on a display.

4. The method of clause 3, wherein the display is configured to receive the input from the patient responsive to the indication presented by the virtual agent.

5. The method of any of clauses 1 to 4, wherein the alarm is indicative of a patient condition.

6. The method of any of clauses 1 to 5, wherein the action is determined via at least one of an alarm rule database, generative artificial intelligence, or machine learning.

7. The method of any of clauses 1 to 6, wherein the patient information is determined from one or more sensors attached to the patient.

8. The method of clause 7, wherein the patient information further includes determining, via the computing device, a position of the patient relative to the first device based on the image data.

9. The method of any of clauses 1 to 8, wherein obtaining the patient information includes at least one of obtaining information about the patient or information from the patient.

10. The method of any of clauses 1 to 9, wherein the image data is captured by at least one of an RGB sensor, a digital camera, an infrared camera, a thermal camera, a depth imaging device, a 3D time of flight camera, or a LIDAR camera.

11. The method of any of clauses 1 to 10, wherein if the alarm persists following an input from the patient responsive to the indication presented by the virtual agent, a second virtual agent is initiated, the second virtual agent configured to present information to a caregiver associated with the patient.

12. The method of clause 11, wherein the second virtual agent acts on instructions provided by the caregiver.

13. A method, including: receiving, by a computing device, first image data of a patient area captured at a first time point, wherein the patient area includes a patient and a plurality of devices associated with the patient; receiving, by the computing device, an indication of an alarm, the alarm being generated by a first device of the plurality of devices associated with the patient; receiving, by the computing device, second image data of the patient area captured at a second time point; determining, by the computing device and based on the second image data, an identity of the first device; determining, by the computing device and based on the first image data, the second image data, and the identity of the first device, a cause of the alarm; determining, by the computing device and based on the cause of the alarm, an action required to resolve the alarm; generating, by the computing device, a virtual agent configured to present an indication of the action; and receiving, by the computing device, an input from the patient responsive to the indication provided by the virtual agent.

14. The method of clause 13, further including causing, by the computing device, a first indication of the action required to resolve the alarm to be presented to the patient via the virtual agent on a display prior to receiving the input from the patient.

15. The method of clause 13 or 14, wherein the action includes at least one of obtaining information about a patient, requesting information from the patient, or requesting that the patient perform a task.

16. The method of clause 15, wherein determining the cause of the alarm further includes comparing the second image data including the first device with expected parameters for the first device.

17. The method of clause 16, wherein receiving, by the computing device, the input from the patient responsive to the virtual agent generates a second indication of the action required to resolve the alarm to be presented to the patient via the virtual agent.

18. The method of any of clauses 13 to 17, wherein the first image data and the second image data is captured by at least one of an RGB sensor, a digital camera, an infrared camera, a thermal camera, a depth imaging device, a 3D time of flight camera, or a LIDAR camera.

19. The method of any of clauses 13 to 19, wherein if the alarm persists following an input from the patient responsive to the indication presented by the virtual agent, a second virtual agent is initiated, the second virtual agent configured to present information to a caregiver associated with the patient.

20. The method of clause 19, wherein the second virtual agent executes instructions provided by the caregiver.

21. A system including: a display including a virtual agent; a plurality of medical devices in a patient area; a rule database; one or more processors communicatively coupled to the display for the virtual agent, and at least one medical device of the plurality of medical devices; and a non-transitory, computer-readable media having instruction stored thereon that, when executed by the one or more processors, cause the one or more processors to perform acts including receiving image data, the image data representing, at least in part, the at least one medical device; determining, using the image data as an input to a first machine learning model, an identification of the at least one medical device; identifying, using the image data as an input to a second machine learning model, an identification of a first state of the at least one medical device; and executing, using the first state of the at least one medical device, a rule from the rule database responsive to the first state of the at least one medical device; wherein executing includes, at least in part, initiation of an interactive virtual agent on the display.

22. The system of clause 21, wherein initiation of the virtual agent includes causing, by a processor of the one or more processors, a first indication of an action required to change the first state of the at least one medical device to be presented to a patient via the virtual agent on the display.

23. The system of clause 21 or 22, wherein the display is configured to receive an input from the patient responsive to the first indication presented by the virtual agent.

24. The system of clause 23, wherein after receiving an input from the patient, the system determines a second state of the at least one medical device.

25. The system of clause any of clauses 21 to 24, wherein the instructions further include retrieving medical records of the patient prior to executing the rule from the rule database.

26. The system of clause 25, wherein the instructions further include obtaining sensor data from sensors attached to the patient prior to executing the rule from the rule database.

27. The system of any of clauses 22 to 26, wherein the first indication is instructions for the patient to change position.

28. The system of any of clauses 22 to 27, wherein the first indication is instructions for the patient to provide spoken information.

29. The system of any of clauses 22 to 28, wherein the image data is captured by at least one of an RGB sensor, a digital camera, an infrared camera, a thermal camera, a depth imaging device, a 3D time of flight camera, or a LIDAR camera.

30. The system of clause 24, wherein if the first indication persists following an input from the patient responsive to the indication presented by the virtual agent, a second virtual agent is initiated, the second virtual agent configured to present information to a caregiver associated with the patient.

31. The system of clause 30, wherein the second virtual agent is configured to execute instructions provided by the caregiver.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components unless the context requires otherwise.

As used herein, the term “based on” can be used synonymously with “based, at least in part, on” and “based at least partly on.”

As used herein, the terms “comprises/comprising/comprised” and “includes/including/included,” and their equivalents can be used interchangeably. An apparatus, system, or method that “comprises A, B, and C” includes A, B, and C, but also can include other components (e.g., D) as well. That is, the apparatus, system, or method is not limited to components A, B, and C.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and website content throughout this specification (referenced materials herein). Each of the referenced materials is individually incorporated herein by reference in their entirety for their referenced teaching(s), as of the filing date of this application.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when the application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8th Edition (Ed. J. Law & R. Rennie, Oxford University Press, 2020).

Although the subject matter has been described in language specific to structural features and/or methodological acts, it is to be understood that the subject matter defined in the appended claims is not necessarily limited to the specific features or acts described.

What is claimed is:

1. A method, comprising:
receiving, by a computing device, an indication of an alarm, the alarm being generated by a first device associated with a patient;
receiving, by the computing device, image data of a patient area, wherein the first device is at least partly disposed in the patient area;
determining, by the computing device, and based on the image data, a cause of the alarm;
determining, by the computing device and based on the cause of the alarm, an action required to resolve the alarm, wherein the action comprises at least one of obtaining patient information or requesting that the patient perform a task;
generating, by the computing device, a virtual agent configured to present an indication of the action;
causing, by the computing device, the indication of the action to be presented to the patient via the virtual agent; and
receiving, by the computing device, an input from the patient responsive to the indication presented by the virtual agent.

2. The method of claim 1, wherein the patient area comprises the patient and a plurality of devices, the method further comprising determining, by the computing device and based on the image data, an identity of the first device in the plurality of devices prior to determining the cause of the alarm.

3. The method of claim 1, wherein the computing device generates the virtual agent on a display.

4. The method of claim 3, wherein the display is configured to receive the input from the patient responsive to the indication presented by the virtual agent.

5. The method of claim 1, wherein the alarm is indicative of a patient condition.

6. The method of claim 1, wherein the action is determined via at least one of an alarm rule database, generative artificial intelligence, or machine learning.

7. The method of claim 1, wherein the patient information is determined from one or more sensors attached to the patient.

8. The method of claim 7, wherein the patient information further comprises determining, via the computing device, a position of the patient relative to the first device based on the image data.

9. The method of claim 1, wherein obtaining the patient information comprises at least one of obtaining information about the patient or information from the patient.

10. The method of claim 1, wherein the image data is captured by at least one of an RGB sensor, a digital camera, an infrared camera, a thermal camera, a depth imaging device, a 3D time of flight camera, or a LIDAR camera.

11. The method of claim 1, wherein if the alarm persists following an input from the patient responsive to the indication presented by the virtual agent, a second virtual agent is initiated, the second virtual agent configured to present information to a caregiver associated with the patient.

12. The method of claim 11, wherein the second virtual agent acts on instructions provided by the caregiver.

13. A method, comprising:
receiving, by a computing device, first image data of a patient area captured at a first time point, wherein the patient area comprises a patient and a plurality of devices associated with the patient;
receiving, by the computing device, an indication of an alarm, the alarm being generated by a first device of the plurality of devices associated with the patient;
receiving, by the computing device, second image data of the patient area captured at a second time point;
determining, by the computing device and based on the second image data, an identity of the first device;
determining, by the computing device and based on the first image data, the second image data, and the identity of the first device, a cause of the alarm;
determining, by the computing device and based on the cause of the alarm, an action required to resolve the alarm;
generating, by the computing device, a virtual agent configured to present an indication of the action; and
receiving, by the computing device, an input from the patient responsive to the indication provided by the virtual agent.

14. The method of claim 13, further comprising causing, by the computing device, a first indication of the action required to resolve the alarm to be presented to the patient via the virtual agent on a display prior to receiving the input from the patient.

15. The method of claim 14, wherein the action includes at least one of obtaining information about a patient, requesting information from the patient, or requesting that the patient perform a task.

16. The method of claim 15, wherein determining the cause of the alarm further comprises comparing the second image data comprising the first device with expected parameters for the first device.

17. The method of claim 16, wherein receiving, by the computing device, the input from the patient responsive to the virtual agent generates a second indication of the action required to resolve the alarm to be presented to the patient via the virtual agent.

18. The method of claim 13, wherein the first image data and the second image data is captured by at least one of an RGB sensor, a digital camera, an infrared camera, a thermal camera, a depth imaging device, a 3D time of flight camera, or a LIDAR camera.

19. The method of claim 13, wherein if the alarm persists following an input from the patient responsive to the indication presented by the virtual agent, a second virtual agent is initiated, the second virtual agent configured to present information to a caregiver associated with the patient.

20. The method of claim 19, wherein the second virtual agent executes instructions provided by the caregiver.
